(19) United States
(12) Patent Application Publication
(10) Pub. No.: US 2026/0007351 A1
(43) Pub. Date: Jan. 8, 2026

(54) EXPERIMENTAL METHOD FOR CLINICAL RESEARCH OF TARGETING MOLECULES BASED ON ATRIAL FIBRILLATION WARNING SYSTEM

(71) Applicant: PLA General Hospital of Northern Theater Command, Shenyang City (CN)

(72) Inventors: Yuji ZHANG, Shenyang City (CN); Huishan WANG, Shenyang City (CN); Yueyang WANG, Shenyang City (CN); Yongbo SONG, Shenyang City (CN); Guannan LIU, Shenyang City (CN); Fangran XIN, Shenyang City (CN)

(21) Appl. No.: 18/830,145

(22) Filed: Sep. 10, 2024

(30) Foreign Application Priority Data
Jul. 4, 2024 (CN) 202410896595.7

(51) Int. Cl. A61B 5/361; A61B 5/00; C12Q 1/6883; G01N 33/68

(52) U.S. Cl. CPC A61B 5/361; A61B 5/746; C12Q 1/6883; G01N 33/6893; C12Q 2600/158; G01N 2570/00; G01N 2800/326; G01N 2800/52

(57) ABSTRACT

This application relates to the technical field of medical research and clinical experiments, and provides an experimental method for clinical research of targeting molecules based on an atrial fibrillation warning system. The method includes: continuously monitoring dynamic electrocardiograms of a patient using an atrial fibrillation warning system; when the atrial fibrillation warning system issues an alert indicating imminent atrial fibrillation, immediately collecting blood or other clinical information of the patient; collecting blood or other clinical information of the patient when the patient has not experienced atrial fibrillation and the atrial fibrillation warning system has not issued an alert, to serve as control data; comparing biological indicators during atrial fibrillation occurrence and non-occurrence, and analyzing potential triggering factors and targeting molecules; and based on the identified targeting molecules, conducting targeted treatment research to validate effectiveness of the targeting molecules.

### CROSS REFERENCE TO RELATED APPLICATION

This patent application claims the benefit and priority of Chinese Patent Application No. 2024108965957, filed with the China National Intellectual Property Administration on Jul. 4, 2024, the disclosure of which is incorporated by reference herein in its entirety as part of the present application.

### TECHNICAL FIELD

The present disclosure relates to the technical field of medical research and clinical experiments, and in particular, to an experimental method for clinical research of targeting molecules based on an atrial fibrillation warning system.

### BACKGROUND

Atrial fibrillation, also known as AFib, is one of the most common types of cardiac arrhythmias. It is characterized by an irregular atrial rhythm caused by multiple small re-entry circuits initiated by the atria. During AFib, the atria are excited at a rate of 300 to 600 beats per minute. The heartbeat frequency is usually high and irregular, sometimes reaching 100 to 160 beats per minute. The heart rate is not only significantly faster compared to normal individuals but also irregular, and the atria lose their effective contraction function. The occurrence of AFib is associated with age and underlying disease types, with hypertension being the most common cardiovascular disease that can lead to AFib. Patients with concomitant AFib have a significantly increased risk of thromboembolic complications.

Atrial fibrillation is a common and dangerous cardiac arrhythmia, especially more prevalent after cardiac surgery. Post-cardiac surgery significantly increases the risk of stroke and medical costs for patients. However, existing technologies lack effective methods for early warning and studying the pathogenesis of atrial fibrillation. Currently, it is challenging to accurately predict the onset of atrial fibrillation, making it difficult to collect clinical samples before and after the onset of the condition for in-depth research, thereby limiting research on targeted treatments for atrial fibrillation.

### SUMMARY

An objective of the present disclosure is to provide an experimental method for clinical research of targeting molecules based on an atrial fibrillation warning system to address the issues raised in the background technology.

To achieve the above objective, the present disclosure provides the following technical solution: an experimental method for clinical research of targeting molecules based on an atrial fibrillation warning system, including the following steps:
S1: warning system monitoring: continuously monitoring dynamic electrocardiograms of a patient using an atrial fibrillation warning system;
S2: alarm-triggered sampling: when the atrial fibrillation warning system issues an alert indicating imminent atrial fibrillation, immediately collecting blood or other clinical information of the patient;
S3: control sampling: when no atrial fibrillation occurs and no alert is issued by the atrial fibrillation warning system, collecting blood or other clinical information of the patient as control samples;
S4: data analysis: comparing biological indicators during atrial fibrillation occurrence and non-occurrence, especially molecular levels in the blood, and analyzing potential triggering factors and targeting molecules; and
S5: targeted treatment research: based on the identified targeting molecules, conducting targeted treatment research to validate effectiveness of the targeting molecules.

Preferably, the warning system monitoring in step S1 includes device selection, monitoring settings, and a monitoring range.

Preferably, the device selection includes selecting a dynamic electrocardiogram recorder that has stable performance and is suitable for long-term wear, such as a portable dynamic electrocardiogram recorder; the monitoring settings include 7-day continuous dynamic electrocardiogram monitoring for the patient, analyzing electrocardiogram data in real time by the atrial fibrillation warning system, and issuing an atrial fibrillation warning 10 to 30 minutes in advance; and the monitoring range covers critical time periods before and after surgery to ensure data integrity and continuity.

Preferably, the alarm-triggered sampling in step S2 includes sampling conditions, sample types, and timing of sampling.

Preferably, the sampling conditions involve immediately notifying a medical team to collect samples when the atrial fibrillation warning system issues an alert indicating imminent atrial fibrillation; the sample types involve collecting venous blood or arterial blood, serum or plasma, or other types of blood samples, while also recording other clinical information of the patient, such as blood pressure, heart rate, respiratory rate, and body temperature; the timing of sampling includes a sampling time point that is set to be as close as possible to a warning time to capture changes in biomarkers indicating imminent atrial fibrillation.

Preferably, the control sampling in step S3 includes sampling conditions; the sampling conditions involve regularly collecting blood or other clinical information when the patient has not experienced atrial fibrillation and the atrial fibrillation warning system has not issued an alert, to serve as control data; timing of control sampling is adjusted based on a daily routine and medical circumstances of the patient to minimize influence of external factors on the control data.

Preferably, the data analysis in step S4 includes an analysis method, biomarker detection, and statistical analysis.

Preferably, the analysis method involves using advanced biochemical and molecular biology techniques, such as proteomics, genomics, and metabolomics, for multi-level analysis of the collected blood samples or other clinical information; the biomarker detection includes measuring molecular levels of proteins, ribonucleic acid (RNA), and metabolites in the blood, or analyzing other clinical data to examine changes before and after the occurrence of atrial fibrillation; the statistical analysis involves performing data processing using a statistical method, to identify significantly different molecules and related biological pathways, and determine the potential triggering factors and targeting molecules.

Preferably, the targeted treatment research in step S5 includes targeting molecule validation and clinical trials; the targeting molecule validation involves designing and conducting targeted treatment research based on the identified targeting molecules to validate effectiveness of the identified targeting molecules in preventing and treating atrial fibrillation; the clinical trials assess safety and efficacy of targeted treatments through animal models or clinical tests, to develop corresponding treatment plans.

Preferably, the targeted treatment research includes validating the safety and efficacy of the targeted treatments in animal models or clinical trials; by comparing various clinical data collected before and after the occurrence of atrial fibrillation and conducting comprehensive analyses of biomarker changes under different conditions, personalized treatment plans are developed, and the experimental method adheres to strict ethical standards during implementation to ensure participant rights and data security.

Compared with the prior art, the present disclosure has the following beneficial effects:

1. The present disclosure includes real-time monitoring and early warning (the atrial fibrillation warning system monitors cardiac activities of a patient in real time and issues alerts in advance to provide an accurate time window for sampling); precise sampling and comparative analysis (samples are collected immediately after an alert, capturing changes in biological indicators before and after the occurrence of atrial fibrillation for accurate comparative analysis); identifying targeting molecules (by analyzing biological indicators under different conditions, potential triggering factors and targeting molecules can be identified, providing a basis for targeted treatments); and improving treatment outcomes (treatment plans designed based on the targeting molecules can effectively prevent and treat atrial fibrillation, improving patient prognosis). The present disclosure can accurately collect and analyze changes in biological indicators before and after the occurrence of atrial fibrillation, identify targeting molecules that trigger atrial fibrillation, and provide scientific support for targeted treatments. This approach not only enhances the precision and reliability of research but also holds significant clinical application value and market potential. Through this system and method, medical personnel can intervene before the occurrence of atrial fibrillation, significantly reducing the incidence of atrial fibrillation and related complications, thereby enhancing the overall treatment outcomes and quality of life for patients.

2. In the present disclosure, blood and other clinical information of the patient are collected after the system issues an alert, and by comparing biological indicators during the occurrence and non-occurrence of atrial fibrillation, particularly the molecular levels in the blood, the targeting molecules that trigger atrial fibrillation are determined, thereby providing a basis for targeted treatments.

### BRIEF DESCRIPTION OF THE DRAWINGS

FIG. 1 is a flowchart of an experimental method for clinical research of targeting molecules based on an atrial fibrillation warning system according to a preferred embodiment of the present disclosure.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All the other embodiments derived by those of ordinary skill in the art based on the embodiments of the present disclosure without creative efforts shall fall within the protection scope of the present disclosure.

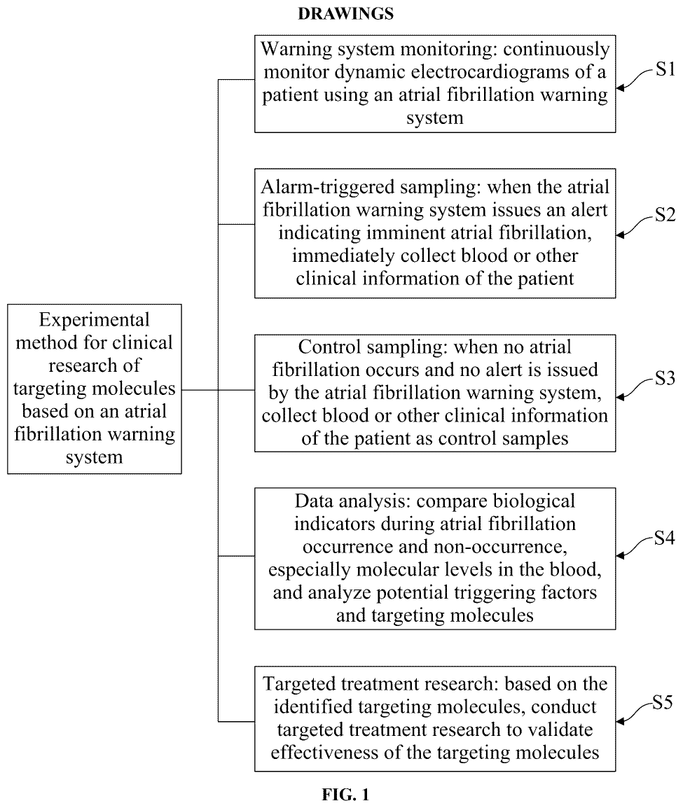

As shown in FIG. 1, an experimental method for clinical research of targeting molecules based on an atrial fibrillation warning system includes the following steps:

S1: Warning system monitoring: continuously monitor dynamic electrocardiograms of a patient using an atrial fibrillation warning system.

The warning system monitoring in step S1 includes device selection, monitoring settings, and detection range. The device selection includes selecting a dynamic electrocardiogram recorder that has stable performance and is suitable for long-term wear, such as a portable dynamic electrocardiogram recorder. The monitoring settings include 7-day continuous dynamic electrocardiogram monitoring for the patient, analyzing electrocardiogram data in real time by the atrial fibrillation warning system, and issuing an atrial fibrillation warning 10 to 30 minutes in advance. The monitoring range covers critical time periods before and after surgery to ensure data integrity and continuity. Before monitoring, it is ensured that all patients sign an informed consent form and fully understand the purpose and process of the monitoring, thereby protecting patient privacy and data security.

S2: Alarm-triggered sampling: when the atrial fibrillation warning system issues an alert indicating imminent atrial fibrillation, immediately collect blood or other clinical information of the patient.

The alarm-triggered sampling in step S2 includes sampling conditions, sample types, and timing of sampling. The sampling conditions involve immediately notifying a medical team to collect samples when the atrial fibrillation warning system issues an alert indicating imminent atrial fibrillation. The sample types involve collecting venous blood or arterial blood, serum or plasma, or other types of blood samples, while also recording other clinical information of the patient, such as blood pressure, heart rate, respiratory rate, and body temperature. The timing of sampling comprises a sampling time point that is set to be as close as possible to a warning time to capture changes in biomarkers indicating imminent atrial fibrillation. The sampling process should be conducted with the informed consent of the patient, ensuring that the sampling procedures comply with medical ethical standards to guarantee patient safety and comfort.

S3: Control sampling: when no atrial fibrillation occurs and no alert is issued by the atrial fibrillation warning system, collect blood or other clinical information of the patient as control samples.

The control sampling in step S3 includes sampling conditions. The sampling conditions involve regularly collecting blood or other clinical information when the patient has not experienced atrial fibrillation and the atrial fibrillation warning system has not issued an alert, to serve as control data. Timing of control sampling is adjusted based on a daily routine and medical circumstances of the patient to minimize influence of external factors on the control data. The collection of control samples should also be conducted with the informed consent of the patient to ensure patient privacy and data security.

S4: Data analysis: compare biological indicators during atrial fibrillation occurrence and non-occurrence, especially molecular levels in the blood, and analyze potential triggering factors and targeting molecules.

The data analysis in step S4 includes an analysis method, biomarker detection, and statistical analysis. The analysis method involves using advanced biochemical and molecular biology techniques, such as proteomics, genomics, and metabolomics, for multi-level analysis of the collected blood samples or other clinical information. The biomarker detection includes measuring molecular levels of proteins, ribonucleic acid (RNA), and metabolites in the blood, or analyzing other clinical data to examine changes before and after the occurrence of atrial fibrillation. The statistical analysis involves performing data processing using a statistical method, to identify significantly different molecules and related biological pathways, and determine the potential triggering factors and targeting molecules. During the data analysis process, patient information should be kept strictly confidential, ensuring that data processing and storage comply with ethical requirements to prevent data leakage.

S5: Targeted treatment research: based on the identified targeting molecules, conduct targeted treatment research to validate effectiveness of the targeting molecules.

The targeted treatment research in step S5 includes targeting molecule validation and clinical trials. The targeting molecule validation involves designing and conducting targeted treatment research based on the identified targeting molecules to validate effectiveness of the identified targeting molecules in preventing and treating atrial fibrillation. The clinical trials assess safety and efficacy of targeted treatments through animal models or clinical tests, to develop corresponding treatment plans. The targeted treatment research includes validating the safety and efficacy of the targeted treatments in animal models or clinical trials; by comparing various clinical data collected before and after the occurrence of atrial fibrillation and conducting comprehensive analyses of biomarker changes under different conditions, personalized treatment plans are developed, and the experimental method adheres to strict ethical standards during implementation to ensure participant rights and data security. Targeted treatment research requires approval from an ethics committee to ensure that the research design and implementation comply with ethical standards, safeguarding participant rights and safety.

The working principle of the present disclosure is as follows: Warning system monitoring is configured, including device selection: selecting a dynamic electrocardiogram recorder that has stable performance and is suitable for long-term wear, such as a portable dynamic electrocardiogram recorder; monitoring settings: conducting 7-day continuous dynamic electrocardiogram monitoring for the patient, analyzing electrocardiogram data in real time by the atrial fibrillation warning system, and issuing an atrial fibrillation warning 10 to 30 minutes in advance; monitoring range: covering critical time periods before and after surgery to ensure data integrity and continuity; and ethical considerations: before monitoring, ensuring that all patients sign an informed consent form and fully understand the purpose and process of the monitoring, thereby protecting patient privacy and data security. Alarm-triggered sampling is configured, including sampling conditions: immediately notifying a medical team to collect samples when the atrial fibrillation warning system issues an alert indicating imminent atrial fibrillation; sample types: collecting venous blood or arterial blood, serum or plasma, or other types of blood samples, while also recording other clinical information of the patient, such as blood pressure, heart rate, respiratory rate, and body temperature; timing of sampling: ensuring that a sampling time point is as close as possible to a warning time to capture changes in biomarkers indicating imminent atrial fibrillation; and ethical considerations: ensuring that the sampling process is conducted with the informed consent of the patient, and ensuring that the sampling procedures comply with medical ethical standards to guarantee patient safety and comfort. Control sampling is configured, including sampling conditions: regularly collecting blood or other clinical information when the patient has not experienced atrial fibrillation and the atrial fibrillation warning system has not issued an alert, to serve as control data, where timing of control sampling is adjusted based on a daily routine and medical circumstances of the patient to minimize influence of external factors on the control data; and ethical considerations: ensuring that collection of control samples is conducted with the informed consent of the patient to guarantee patient privacy and data security. Data analysis is configured, including an analysis method: using advanced biochemical and molecular biology techniques, such as proteomics, genomics, and metabolomics, for multi-level analysis of the collected blood samples or other clinical information; biomarker detection: measuring molecular levels of proteins, RNA, and metabolites in the blood, or analyzing other clinical data to examine changes before and after the occurrence of atrial fibrillation; statistical analysis: performing data processing using a statistical method, to identify significantly different molecules and related biological pathways, and determine the potential triggering factors and targeting molecules; and ethical considerations: during the data analysis process, ensuring that patient information is kept strictly confidential, to guarantee that data processing and storage comply with ethical requirements to prevent data leakage. Targeted treatment research is configured, including: targeting molecule validation: designing and conducting targeted treatment research based on the identified targeting molecules to validate effectiveness of the identified targeting molecules in preventing and treating atrial fibrillation; clinical trials: assessing safety and efficacy of targeted treatments through animal models or clinical tests, to develop corresponding treatment plans; and ethical considerations: targeted treatment research receives requires approval from an ethics committee to ensure that the research design and implementation comply with ethical standards, safeguarding participant rights and safety. The atrial fibrillation warning system monitors cardiac activities of a patient in real time and issues alerts in advance to provide an accurate time window for sampling. Through precise sampling and comparative analysis, samples are collected immediately after an alert, capturing changes in biological indicators before and after the occurrence of atrial fibrillation for accurate comparative analysis. Targeting molecules are identified: by analyzing biological indicators under different conditions, potential triggering factors and targeting molecules can be identified, providing a basis for targeted treatments. Treatment outcomes are improved: treatment plans designed based on the targeting molecules can effectively prevent and treat atrial fibrillation, improving patient prognosis.

It should be noted that relational terms herein such as first and second are merely used to distinguish one entity or operation from another entity or operation without necessarily requiring or implying any actual such relationship or order between such entities or operations. In addition, terms “include”, “comprise”, or their any other variations are intended to cover a non-exclusive inclusion, such that a process, a method, an article, or a device that includes a series of elements not only includes those elements, but also includes other elements that are not explicitly listed, or also includes inherent elements of the process, the method, the article, or the device. In the case that there are no more restrictions, an element limited by the statement “includes a . . . ” does not exclude the presence of additional identical elements in the process, the method, the article, or the device that includes the element.

Although the embodiments of the present disclosure have been illustrated and described, it should be understood that those of ordinary skill in the art may make various changes, modifications, replacements and variations to the above embodiments without departing from the principle and spirit of the present disclosure, and the scope of the present disclosure is limited by the appended claims and their legal equivalents.

What is claimed is:

1. An experimental method for clinical research of targeting molecules based on an atrial fibrillation warning system, comprising the following steps:
S1: warning system monitoring: continuously monitoring dynamic electrocardiograms of a patient using an atrial fibrillation warning system;
S2: alarm-triggered sampling: when the atrial fibrillation warning system issues an alert indicating imminent atrial fibrillation, immediately collecting blood or other clinical information of the patient;
S3: control sampling: when no atrial fibrillation occurs and no alert is issued by the atrial fibrillation warning system, collecting blood or other clinical information of the patient as control samples;
S4: data analysis: comparing biological indicators during atrial fibrillation occurrence and non-occurrence, especially molecular levels in the blood, and analyzing potential triggering factors and targeting molecules; and
S5: targeted treatment research: based on the identified targeting molecules, conducting targeted treatment research to validate effectiveness of the targeting molecules.

2. The experimental method for clinical research of targeting molecules based on an atrial fibrillation warning system according to claim 1, wherein the warning system monitoring in step S1 comprises device selection, monitoring settings, and a monitoring range.

3. The experimental method for clinical research of targeting molecules based on an atrial fibrillation warning system according to claim 2, wherein the device selection comprises selecting a dynamic electrocardiogram recorder that has stable performance and is suitable for long-term wear, specifically comprising a portable dynamic electrocardiogram recorder; the monitoring settings comprise 7-day continuous dynamic electrocardiogram monitoring for the patient, analyzing electrocardiogram data in real time by the atrial fibrillation warning system, and issuing an atrial fibrillation warning 10 to 30 minutes in advance; and the monitoring range covers critical time periods before and after surgery to ensure data integrity and continuity.

4. The experimental method for clinical research of targeting molecules based on an atrial fibrillation warning system according to claim 1, wherein the alarm-triggered sampling in step S2 comprises sampling conditions, sample types, and timing of sampling.

5. The experimental method for clinical research of targeting molecules based on an atrial fibrillation warning system according to claim 4, wherein the sampling conditions involve immediately notifying a medical team to collect samples when the atrial fibrillation warning system issues an alert indicating imminent atrial fibrillation; the sample types involve collecting venous blood or arterial blood, serum or plasma, or other types of blood samples, while also recording other clinical information of the patient, comprising blood pressure, heart rate, respiratory rate, and body temperature; and the timing of sampling comprises a sampling time point that is set to be as close as possible to a warning time to capture changes in biomarkers indicating imminent atrial fibrillation.

6. The experimental method for clinical research of targeting molecules based on an atrial fibrillation warning system according to claim 1, wherein the control sampling in step S3 comprises sampling conditions; the sampling conditions involve regularly collecting blood or other clinical information when the patient has not experienced atrial fibrillation and the atrial fibrillation warning system has not issued an alert, to serve as control data; and timing of control sampling is adjusted based on a daily routine and medical circumstances of the patient to minimize influence of external factors on the control data.

7. The experimental method for clinical research of targeting molecules based on an atrial fibrillation warning system according to claim 1, wherein the data analysis in step S4 comprises an analysis method, biomarker detection, and statistical analysis.

8. The experimental method for clinical research of targeting molecules based on an atrial fibrillation warning system according to claim 7, wherein the analysis method involves using advanced biochemical and molecular biology techniques comprising proteomics, genomics, and metabolomics, for multi-level analysis of the collected blood samples or other clinical information; the biomarker detection comprises measuring molecular levels of proteins, ribonucleic acid (RNA), and metabolites in the blood, or analyzing other clinical data to examine changes before and after the occurrence of atrial fibrillation; the statistical analysis involves performing data processing using a statistical method, to identify significantly different molecules and related biological pathways, and determine the potential triggering factors and targeting molecules.

9. The experimental method for clinical research of targeting molecules based on an atrial fibrillation warning system according to claim 1, wherein the targeted treatment research in step S5 comprises targeting molecule validation and clinical trials; the targeting molecule validation involves designing and conducting targeted treatment research based on the identified targeting molecules to validate effectiveness of the identified targeting molecules in preventing and treating atrial fibrillation; the clinical trials assess safety and efficacy of targeted treatments through animal models or clinical tests, to develop corresponding treatment plans.

10. The experimental method for clinical research of targeting molecules based on an atrial fibrillation warning system according to claim 9, wherein the targeted treatment research comprises validating the safety and efficacy of the targeted treatments in animal models or clinical trials; by comparing various clinical data collected before and after the occurrence of atrial fibrillation and conducting comprehensive analyses of biomarker changes under different conditions, personalized treatment plans are developed, and the experimental method adheres to strict ethical standards during implementation to ensure participant rights and data security.
